(19) United States
(12) Patent Application Publication
(10) Pub. No.: US 2023/0014293 A1
(43) Pub. Date: Jan. 19, 2023

(54) AQUEOUS, ROOM-TEMPERATURE STABLE ROCURONIUM COMPOSITION

(71) Applicant: FRESENIUS KABI AUSTRIA GMBH, Graz (AT)

(72) Inventors: Christian MASSER, Graz (AT); Birgit STEINER-ZITZENBACHER, Graz (AT); Theofanis MANTOURLIAS, Graz (AT)

(73) Assignee: FRESENIUS KABI AUSTRIA GMBH, Graz (AT)

(21) Appl. No.: 17/406,771

(22) Filed: Aug. 19, 2021

(30) Foreign Application Priority Data
Jul. 2, 2021 (EP) 21183555.8

(51) Int. Cl. A61K 31/58; A61K 47/02; A61K 9/08; A61J 1/20

(52) U.S. Cl. CPC A61K 31/58; A61K 47/02; A61K 9/08; A61J 1/2096

(57) ABSTRACT

The invention relates to a rocuronium composition providing no or less vascular pain to patients upon injection. The rocuronium composition has a pH of 2.5 to 3.5 and a titratable acidity of not more than 35 mEq. The rocuronium composition is stable upon thermal sterilization. Further, the rocuronium composition is stable upon storage at room-temperature.

### CROSS-REFERENCE TO RELATED APPLICATIONS

This patent application claims the benefit of European Patent Application No. 21183555.8, filed on Jul. 2, 2021, the disclosure of which is incorporated herein by reference in its entirety for all purposes.

### FIELD OF THE INVENTION

The invention relates to an injectable, room-temperature stable rocuronium composition, to a process for preparing the composition, as well as to a container comprising the composition.

### BACKGROUND OF THE INVENTION

Rocuronium is a neuromuscular blocking agent which is used in order to induce muscle relaxation during anesthesia. The rapid onset and short-acting behavior of neuromuscular blocking agents are key features to the success of these drugs. Typically, neuromuscular blocking agents are applied by intravenous injection.

Rocuronium (or rocuronium bromide) has the following structural formula I:

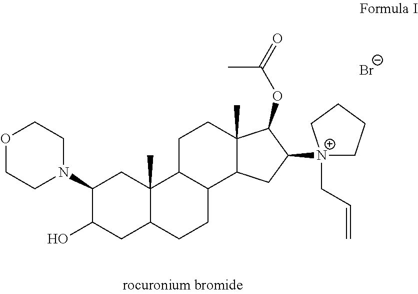 [SMILES: C=CC[N+]1([C@H]2CC3C4CCC5CC(O)[C@@H](N6CCOCC6)C[C@]5(C)C4CC[C@]3(C)[C@H]2OC(C)=O)CCCC1 ; [Br-]]

There are several commercially available drug products comprising rocuronium. One of these products is sold by Merck, Sharp & Dohme under the tradenames ESMERON™, ESLAX™, and ZEMURON™ as an aqueous solution for injection comprising 10 mg/ml rocuronium and sodium acetate buffer with a pH of 3.8 to 4.2. This product is thermally unstable and thus requires cold-chain transportation and storage at 2° to 8° C. The package insert for ESMERON™ states that storage outside of the refrigerator at a temperature of up to 30° C. shall not exceed 12 weeks, which can be inconvenient for health care providers and costly to manufacturers, distributors, payors, and end users. EP3017817A1 (originally published as WO 2015/001995) discloses that the commercial ESLAX™ product comprising 10 mg/ml rocuronium and an acetate buffer at pH 4.0 has a titratable acidity of 114 mEq.

Another commercially available drug product is Rocuronium Bromide Intravenous Solution/MR13A10A sold by Maruishi Pharmaceuticals in Japan as an aqueous solution for injection comprising 10 mg/ml rocuronium, 0.5% sodium chloride, and 0.55% glycine buffer with a pH of 3 (Shimizu, PLoS One 14(10): e0223947). EP3017817A1 discloses that preparations comprising 10 mg/ml rocuronium and a glycine-hydrochloric acid buffer at pH 3.0 have a titratable acidity of 40 mEq and 79 mEq at hydrochloric acid concentrations of 0.045M and 0.09M, respectively. EP3017817A1 also discloses buffered rocuronium preparations having a titratable acidity of between 39 mEq (tartrate, formate) and 83 mEq (citrate), depending on the buffer used and its concentration. EP3017817A1 discloses that vascular pain in a rat pain model is reduced by administration of buffered rocuronium preparations having a titratable acidity of 100 mEq or less and a pH of 2.5 to 4.5.

Several other pharmaceutical compositions of rocuronium have been described.

For example, WO 2008/065142 describes a pharmaceutical composition in the form of an aqueous solution for parenteral administration comprising rocuronium and a sulfoalkylether-beta-cyclodextrin derivative or a pharmaceutically acceptable salt thereof for stability improvement. The compositions described in WO 2008/065142 preferably are isotonic, may comprise a buffer, and have a pH of 3.5 to 7.5, or a pH of 5.5 to 7.5.

EP2900216B1 (originally published as WO 2014/048836) describes an aqueous composition comprising a rocuronium salt and a stabilizing excipient selected from D-gluconic acid, an intramolecular lactone of D-gluconic acid and a mixture thereof. The compositions described in EP2900216B1 may comprise a buffer and have a pH of 7 or below, or a pH of about 3.8 to about 4.0.

EP3162370A1 (originally published as WO 2015/198456) describes rocuronium formulations comprising rocuronium and a buffer solution with a pH of 3.5 or less, in particular with a pH of 2.5 to 3.5.

However, there is still the need for aqueous rocuronium compositions that are stable at room temperature with an acceptable shelf-life and which provide a tolerable pain upon injection.

### SUMMARY OF THE INVENTION

The inventors have surprisingly found that an aqueous rocuronium composition with a pH of 2.5 to 3.5 and a titratable acidity of not more than 35 mEq is stable upon thermal sterilization and has an estimated shelf-life at room temperature of several years, preferably at least three years.

Thus, the present invention relates to an aqueous, room-temperature stable composition comprising rocuronium, wherein the composition has a pH of 2.5 to 3.5, and wherein the composition has a titratable acidity of not more than 35 mEq.

The present invention also relates to a container comprising the aqueous, room-temperature stable composition comprising rocuronium, wherein the composition has a pH of 2.5 to 3.5, and wherein the composition has a titratable acidity of not more than 35 mEq.

The present invention also relates to a process for preparing the aqueous composition according to the present invention comprising the steps of:

a. Dissolving a tonicity agent (e.g., NaCl) in water,

b. Adding of HCl to adjust the pH to 1.6 to 2.0,

c. Adding and dissolving rocuronium,

d. Adding HCl to adjust the pH to 2.5 to 3.5,
and

e. Filling the composition into a container,

wherein sterility is brought about either by thermal sterilization or by aseptic filling.

### DETAILED DESCRIPTION OF THE INVENTION

The present invention relates to an aqueous, room-temperature stable composition comprising rocuronium, wherein the composition has a pH of 2.5 to 3.5, and wherein the composition has a titratable acidity of not more than 35 mEq.

The pH of the aqueous, room-temperature stable composition of the present invention is in the range of 2.5 to 3.5. Preferably the composition according to the present invention has a pH of 2.6 to 3.4, more preferably a pH of 2.7 to 3.3, even more preferably a pH of 2.8 to 3.2, even more preferably a pH of 2.9 to 3.1, and most preferred a pH of 3.0.

The pH of the aqueous, room-temperature stable composition of the present invention may be adjusted by adding alkalizing and/or acidifying agents. Preferably, the alkalizing agent is selected from the group consisting of sodium carbonate, potassium carbonate, sodium bicarbonate, potassium bicarbonate, sodium hydroxide and potassium hydroxide, most preferably the alkalizing agent is sodium hydroxide. Preferably, the acidifying agent is hydrochloric acid (HCl), more preferably a 0.01 to 10 M (mol/l) aqueous solution of HCl, even more preferably a 0.1 to 5 M aqueous solution of HCl, e.g. a 1 M aqueous solution of HCl or a 5 M aqueous solution of HCl.

Preferably, the pH of the composition of the present invention is adjusted by adding an aqueous solution, preferably an 0.01 to 10 M (mol/l) aqueous solution of HCl, more preferably a 0.1 to 5 M aqueous solution of HCl, e.g. a 1 M aqueous solution of HCl or a 5 M aqueous solution of HCl.

The pH of the aqueous, room-temperature stable composition of the present invention preferably does not drift upon sterilization or storage. Preferably, the drift upon sterilization or storage of the aqueous, room-temperature stable composition of the present invention is not more than 0.5 pH units, more preferably not more than 0.4 pH units, even more preferably not more than 0.3 pH units, even more preferably not more than 0.2 pH units, and most preferably not more than 0.1 pH units.

The pH of the aqueous, room-temperature stable composition of the present invention preferably does not drift upon sterilization. Preferably, the drift upon sterilization of the aqueous, room-temperature stable composition of the present invention is not more than 0.5 pH units, more preferably not more than 0.4 pH units, even more preferably not more than 0.3 pH units, even more preferably not more than 0.2 pH units, and most preferably not more than 0.1 pH units.

The pH of the aqueous, room-temperature stable composition of the present invention preferably does not drift upon storage. Preferably, the drift upon storage of the aqueous, room-temperature stable composition of the present invention is not more than 0.5 pH units, more preferably not more than 0.4 pH units, even more preferably not more than 0.3 pH units, even more preferably not more than 0.2 pH units, and most preferably not more than 0.1 pH units.

The pH of the aqueous, room-temperature stable composition of the present invention preferably does not drift upon sterilization and storage. Preferably, the drift upon sterilization and storage of the aqueous, room-temperature stable composition of the present invention is not more than 0.5 pH units, more preferably not more than 0.4 pH units, even more preferably not more than 0.3 pH units, even more preferably not more than 0.2 pH units, and most preferably not more than 0.1 pH units.

The pH of the aqueous composition of the present invention as used herein refers to the pH value measured at room temperature.

The term “thermal sterilization” as used herein refers to heat sterilization, preferably to moist heat sterilization. Preferably, moist heat sterilization is used with overheated water as sterilizing medium. The temperature of the overheated water is generally at least 100° C., preferably at least 110° C., more preferably at least 120° C. The pressure during thermal sterilization is generally at least 1 bar (100 kilopascal), for example at least 1.5 bar (150 kilopascal), at least 1.7 bar (170 kilopascal), at least 2 bar (200 kilopascal), at least 3 bar (300 kilopascal), or at least 4 bar (400 kilopascal). In some embodiments, the pressure during thermal sterilization is between 1 bar and 4 bar, e.g., 1-3 bar, 1.5-2 bar, 1.7-2 bar, or 1.7-3 bar.

Thermal sterilization is generally carried out for at least 10 minutes, preferably for at least 15 minutes, more preferably at least 20 minutes.

Preferably, the thermal sterilization of the aqueous, room-temperature stable composition according to the present invention is carried out at a temperature of 120° C.-122° C. and a pressure of 2 bar (200 kilopascal) for 15-20 minutes, more preferably the thermal sterilization is carried out at a temperature of 121° C. and a pressure of 2 bar (200 kilopascal) for 15 minutes.

The titratable acidity of the aqueous, room-temperature stable composition according to the present invention is not more than 35 mEq. Preferably, the titratable acidity of the aqueous, room-temperature stable composition according to the present invention is not more than 30 mEq, more preferably not more than 25 mEq, even more preferably not more than 20 mEq, and most preferred not more than 10 mEq. In some embodiments, the titratable acidity of the composition is between 3 mEq and 35 mEq, e.g., 5-30 mEq, 3-20 mEq, 10-25 mEq, 8-20 mEq, 3-10 mEq, or 5-10 mEq.

The term “titratable acidity” as used herein refers to the quantity (mEq) of sodium hydroxide consumed in the titration of 1 L of a solution to pH 7.4.

Titratable acidity as used herein refers to the titratable acidity measured at room temperature.

The aqueous composition of the present invention is stable at room-temperature. The European Pharmacopoeia, Edition 10.3 defines the term “room temperature” as a temperature in the range of 15° C.-25° C. The United States Pharmacopoeia, Edition USP43-NF38 2S defines “controlled room temperature” as a temperature between 20° C.-25° C.

In the context of the present invention the term “room temperature” refers to a temperature range of 20° C.-25° C.

The term “room-temperature stable” as used herein refers to the stability of rocuronium in the aqueous composition at temperatures between 20° C. and 25° C. During storage the concentration of rocuronium decreases due to degradation of rocuronium. The main degradation product of rocuronium is “impurity C”, also referred to as “rocuronium-related impurity C” or “des-17-acetyl rocuronium”, of structural formula II, which is formed by hydrolysis of rocuronium:

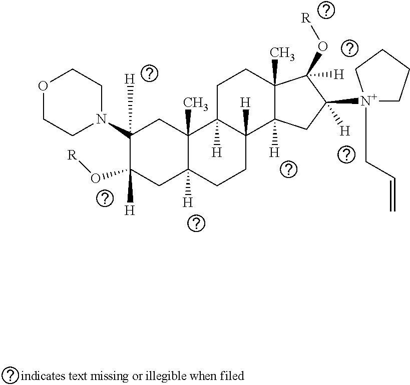

Formula II: Rocuronium-related impurity C=des-17-acetyl rocuronium; wherein R═R′═H

Thus, during storage the concentration of rocuronium decreases and the concentration of rocuronium-related impurity C increases. The hydrolysis of rocuronium is temperature dependent. For example, the percentage of “rocuronium-related impurity C” in a composition will be higher if—for a defined period of time—the composition is stored at 40° C. than if it is stored at 25° C.

The concentration of “rocuronium-related impurity C” in the aqueous composition as used herein refers to the concentration of “rocuronium-related impurity C” determined by high performance liquid chromatography (HPLC) based on the method described in the Pharm. Eu. 10.3 Monograph for Rocuronium Bromide (1764) as described in example 2.b.1.

“Rocuronium-related impurity C” is measured as percentage of rocuronium in the sample at the time point of measurement. For example, a value of 0.67% rocuronium-related impurity C means that the area under the chromatographic curve of rocuronium-related impurity C is 0.67% of the area under the chromatographic curve of rocuronium.

Preferably, the composition according to the present invention does not exhibit more than 0.7%, more preferred not more than 0.6%, and most preferred not more than 0.5%, rocuronium-related impurity C after storage for 6 months at room temperature. In some embodiments, the amount of rocuronium-related impurity C present in the composition after storage for 6 months at room temperature is between 0.1% and 0.7%, e.g., 0.1-0.6%, 0.2-0.5%, 0.4-0.7% or 0.3-0.6%.

Preferably, the composition according to the present invention does not exhibit more than 1.1%, more preferred not more than 1.0%, even more preferred not more than 0.9%, and most preferred not more than 0.8%, rocuronium-related impurity C after storage for 12 months at room temperature. In some embodiments, the amount of rocuronium-related impurity C present in the composition after storage for 12 months at room temperature is between 0.2% and 1.1%, e.g., 0.2-0.8%, 0.2-0.7%, 0.4-0.8% or 0.3-0.6%.

Preferably, the composition according to the present invention does not exhibit more than 1.1%, more preferred not more than 1.0%, even more preferred not more than 0.9%, and most preferred not more than 0.8%, rocuronium-related impurity C after storage for 6 months at 30+/−2° C. In some embodiments, the amount of rocuronium-related impurity C present in the composition after storage for 6 months at 30+/−2° C. is between 0.2% and 1.1%, e.g., 0.2-0.8%, 0.2-0.7%, 0.4-0.8% or 0.3-0.6%.

Preferably, the composition according to the present invention does not exhibit more than 1.6%, more preferred not more than 1.5%, even more preferred not more than 1.4%, and most preferred not more than 1.3%, rocuronium-related impurity C after storage for 12 months at 30+/−2° C. In some embodiments, the amount of rocuronium-related impurity C present in the composition after storage for 12 months at 30+/−2° C. is between 0.6% and 1.6%, e.g., 0.6-1.4%, 0.7-1.3%, 0.8-1.3% or 0.9-1.3%.

Preferably, the composition according to the present invention does not exhibit more than 2.3%, more preferred not more than 2.2%, even more preferred not more than 2.1%, even more preferred not more than 2.0%, even more preferred not more than 1.9%, and most preferred not more than 1.8%, rocuronium-related impurity C after storage for 6 months at 40+/−2° C. In some embodiments, the amount of rocuronium-related impurity C present in the composition after storage for 6 months at 40+/−2° C. is between 0.5% and 2.3%, e.g., 0.8-2.1%, 0.9-2.0%, 1.0-1.9% or 1.2-1.8%.

The aqueous, room-temperature stable composition of the present invention has excellent storage stability when stored in a pharmaceutical container. The pharmaceutical container may be an ampoule, a bottle, a bag, a cartridge, a syringe, or a vial. Preferably, the container is a syringe or a vial.

The container may comprise glass or a synthetic polymer. Preferably the synthetic polymer is an organic polymer. Preferably, the organic polymer comprises a polyethylene, a polypropylene, a cyclic olefin polymer or a cyclic olefin copolymer. One or more surfaces of the container can be treated with a compound to limit reactivity with one or more components of the composition of the invention. In some embodiments, the container is treated with silicone. In other embodiments, the container is treated with a sulfur-containing compound. In yet other embodiments, the container is not treated.

In one embodiment, the container may be a vial. Preferably, the vial is a glass vial or a plastic vial, more preferably the vial is a glass vial. The glass vial may either be a transparent glass vial or a light protective glass vial, preferably a transparent glass vial.

The aqueous, room-temperature stable composition of the present invention has excellent storage stability when stored in a glass vial.

In a further embodiment, the container may be a syringe. Preferably, the syringe is a glass syringe or a syringe made of material comprising a synthetic polymer, more preferably a syringe made of material comprising a synthetic polymer. Particularly preferred, the syringe material comprises an organic polymer, preferably a polyethylene, a polypropylene, a cyclic olefin polymer or a cyclic olefin copolymer. Most preferably the syringe material comprises a cyclic olefin copolymer.

The pharmaceutical container is sealed by way of a closure, such as a stopper, valve, plunger, and/or tip cap. Preferably, the closure is made with an inert material such as rubber or plastic. In some embodiments, the closure is coated with a silicone polymer or a fluoropolymer. In other embodiments, the closure is not coated. Not limiting examples of suitable closures comprise bromobutyl rubber, chlorobutyl rubber, and coated versions thereof.

In some embodiments, the composition according to the present invention does not exhibit more than 0.7%, more preferred not more than 0.6%, and most preferred not more than 0.5%, rocuronium-related impurity C after storage for 6 months at room temperature when stored in a glass vial. In some embodiments, the amount of rocuronium-related impurity C present in the composition after storage for 6 months at room temperature in a glass vial is between 0.1% and 0.7%, e.g., 0.1-0.6%, 0.2-0.5%, 0.4-0.7% or 0.3-0.6%.

In other embodiments, the composition according to the present invention does not exhibit more than 1.1%, more preferred not more than 1.0%, even more preferred not more than 0.9%, and most preferred not more than 0.8%, rocuronium-related impurity C after storage for 12 months at room temperature when stored in a glass vial. In some embodiments, the amount of rocuronium-related impurity C present in the composition after storage for 12 months at room temperature in a glass vial is between 0.2% and 1.1%, e.g., 0.2-0.8%, 0.2-0.7%, 0.4-0.8% or 0.3-0.6%.

The aqueous, room-temperature stable composition of the present invention also has excellent storage stability when stored in a syringe, such as a syringe made of a material comprising a cyclic olefin copolymer.

The composition according to the present invention does not exhibit more than 0.6%, more preferred not more than 0.5%, and most preferred not more than 0.4%, rocuronium-related impurity C after storage for 6 months at room temperature when stored in a syringe. In some embodiments, the amount of rocuronium-related impurity C present in the composition after storage for 6 months at room temperature in a syringe is between 0.1% and 0.7%, e.g., 0.1-0.6%, 0.2-0.4%, or 0.3-0.5%.

The composition according to the present invention does not exhibit more than 0.8%, more preferred not more than 0.7%, and most preferred not more than 0.6%, rocuronium-related impurity C after storage for 12 months at room temperature when stored in a syringe. In some embodiments, the amount of rocuronium-related impurity C present in the composition after storage for 12 months at room temperature in a syringe is between 0.2% and 1.0%, e.g., 0.2-0.8%, 0.4-0.7% or 0.3-0.6%.

As the aqueous composition according to the present invention is to be administered parenterally, it comprises water in a purity suitable for parenteral administration, i.e. water for injection (WFI).

The aqueous composition of the present invention comprises rocuronium, preferably in form of a pharmaceutically acceptable salt, more preferably in form of the bromide salt (Formula I). The composition preferably contains a therapeutically effective amount of rocuronium or pharmaceutically acceptable salt thereof. In some embodiments, the composition comprises 1-50 mg/mL of rocuronium or pharmaceutically acceptable salt thereof. In preferred embodiments, the composition comprises 10 mg/mL rocuronium or pharmaceutically acceptable salt thereof.

The aqueous, room-temperature stable composition of the present invention preferably does not comprise a buffer. The aqueous, room-temperature stable composition of the present invention preferably does not comprise a stabilizing excipient, such as a cyclodextrin (e.g., sulfobutylether β-cyclodextrin) or a polyhydroxy acid (e.g., D-gluconic acid) or intramolecular lactone thereof.

The aqueous, room-temperature stable composition of the present invention may further comprise a tonicity agent and hydrochloric acid. Suitable tonicity agents include, without limitation, sodium chloride, dextrose, mannitol, trehalose, potassium chloride, and glycerol. Preferably, the tonicity agent is sodium chloride or dextrose. More preferably, the tonicity agent is sodium chloride.

In a preferred embodiment, the aqueous, room-temperature stable composition of the present invention comprises rocuronium bromide, sodium chloride, hydrochloric acid, water, and optionally sodium hydroxide for pH adjustment.

In another preferred embodiment, the aqueous, room-temperature stable composition of the present invention consists essentially of rocuronium bromide, sodium chloride, hydrochloric acid, water, and optionally sodium hydroxide for pH adjustment.

In yet another preferred embodiment, the aqueous, room-temperature stable composition of the present invention consists of rocuronium bromide, sodium chloride, hydrochloric acid and water.

The term “consist of” or “consists essentially of” as used herein includes any impurities of the ingredients.

The present invention also relates to a container comprising the aqueous, room-temperature stable composition comprising rocuronium, wherein the composition has a pH of 2.5 to 3.5, and wherein the composition has a titratable acidity of not more than 35 mEq.

The present invention also relates to a process for preparing the aqueous, room-temperature stable composition according to the invention.

The aqueous, room-temperature stable composition of the present invention can be prepared according to processes known by a person skilled in the art. For example, the aqueous, room-temperature stable composition of the present invention can be prepared by a) mixing its components and b) pH adjustment. In particularly preferred embodiments, the aqueous, room-temperature stable composition of the present invention the process for preparing the aqueous composition comprises the steps of:

a. Dissolving a tonicity agent (e.g., NaCl) in water,

b. Adding of HCl to adjust the pH to 1.6 to 2.0,

c. Adding and dissolving rocuronium,

d. Adding HCl to adjust the pH to 2.5 to 3.5,
and

e. Filling the composition into a container,

wherein sterility is brought about either by thermal sterilization or by aseptic filling.

The process can comprise a step of filtering the composition prior to filling into the container. For filtration, filters with a pore size in the range of 0.2 to 0.6 μm can for instance be used, capable of removing microbiological contaminations. Preferably, the filter has a pore size of 0.2 μm.

The filling of the containers is done using filling techniques known in the art.

The aqueous, room-temperature stable composition of the present invention is sterilized. Typical sterilization methods include sterilization by dry heat, moist heat, irradiation and gas exposure. Sterilization of the composition according to the present invention preferably is performed either by thermal sterilization or by aseptic filling.

Where the room-temperature stable composition of the present invention is filled into and stored within a glass vial, the composition can either be sterilized by thermal sterilization or it may be filled aseptically. Preferably, the composition is sterilized by thermal sterilization, more preferably by moist heat sterilization with overheated water as sterilizing medium. The temperature of the overheated water is generally at least 100° C., preferably at least 110° C., more preferably at least 120° C. The pressure during thermal sterilization is generally at least 1 bar (100 kilopascal), for example at least 1.5 bar (150 kilopascal), at least 1.7 bar (170 kilopascal), at least 2 bar (200 kilopascal), at least 3 bar (300 kilopascal), or at least 4 bar (400 kilopascal). In some embodiments, the pressure during thermal sterilization is between 1 bar and 4 bar, e.g., 1-3 bar, 1.5-2 bar, 1.7-2 bar, or 1.7-3 bar.

In a particularly preferred embodiment, the container, preferably being a glass vial, comprising the aqueous, room-temperature stable composition of the present invention is heat sterilized at 120°-122° C. and a pressure of 2 bar (200 kilopascal) for 15-20 minutes.

Where the room-temperature stable composition of the invention is filled into and stored within a polymeric syringe as container, thermal sterilization may be impractical, such that the composition may be filled aseptically.

The terms “filled aseptically”, “aseptically filled” and “aseptic filling” as used herein mean that the container and the composition are pre-sterilized prior to filling the composition into the container. Preferably, the syringe is pre-sterilized via gamma irradiation or via treatment with ethylene oxide. The composition is filtered through at least one, preferably through at least two filters with a pore size of 0.22 μm or less prior to filling the composition into the syringe. Filtering the composition and pre-sterilizing the syringe are preferably performed in a sterile chamber.

According to one aspect of the invention the aqueous, room-temperature stable composition according to the invention is for use as a medicament. Preferably, it is for use in general anesthesia to facilitate tracheal intubation during routine sequence induction or to provide skeletal muscle relaxation during surgery or to facilitate tracheal intubation during rapid sequence induction or for short term use in an intensive care unit.

The invention also provides a method of facilitating tracheal intubation during general anesthesia by administering the composition of the invention to a patient in need thereof. In another aspect, the invention provides a method of providing skeletal muscle relaxation during surgery or mechanical ventilation by administering the composition of the invention to a patient in need thereof.

The aqueous, room-temperature stable composition according to the invention is preferably for parenteral administration, most preferably for intravenous administration.

### Embodiments

1. An aqueous, room-temperature stable composition comprising rocuronium, wherein the composition has a pH of 2.5 to 3.5, and wherein the composition has a titratable acidity of not more than 35 mEq.

2. The composition according to embodiment 1, wherein the composition has a pH of 2.8 to 3.2, preferably a pH of 3.0.

3. The composition according to embodiment 1 or 2, wherein the drift of the pH following sterilization or storage of the composition is not more than 0.5 pH units, preferably not more than 0.2 pH units.

4. The composition according to embodiment 1 to 3, wherein thermal sterilization is carried out at a temperature of 120 to 122° C. for 15 to 20 minutes.

5. The composition according to any of embodiments 1 to 4, wherein the composition does not exhibit more than 0.7%, preferably not more than 0.5%, rocuronium-related impurity C after storage for 6 months at room temperature.

6. The composition according to any of embodiments 1 to 5, wherein the composition does not exhibit more than 1.1%, preferably not more than 0.8%, rocuronium-related impurity C after storage for 12 months at room temperature.

7. The composition according to any of embodiments 1 to 6, wherein the composition does not exhibit more than 1.0%, preferably not more than 0.8%, rocuronium-related impurity C after storage for 6 months at 30° C.

8. The composition according to embodiments 1 to 7, wherein the composition does not exhibit more than 2.0%, preferably not more than 1.8% rocuronium-related impurity C after storage for 6 months at 40° C.

9. The composition according to any of embodiments 1 to 8, wherein the composition does not comprise a buffer.

10. The composition according to any of embodiments 1 to 9, wherein the composition has a titratable acidity of not more than 30 mEq, preferably of not more than 25 mEq, most preferred of not more than 20 mEq.

11. The composition according to any of embodiments 1 to 10, wherein the composition comprises rocuronium in the form of rocuronium bromide.

12. The composition according to any of embodiments 1 to 11, wherein the composition further comprises a tonicity agent, preferably sodium chloride, and hydrochloric acid.

13. The composition according to any of embodiments 1 to 12 for use as a medicament.

14. The composition for use according to embodiment 13 for use in general anesthesia to facilitate tracheal intubation during routine sequence induction or to provide skeletal muscle relaxation during surgery or to facilitate tracheal intubation during rapid sequence induction or for short term use in the intensive care unit.

15. Container comprising the composition according to any of embodiments 1 to 12.

16. Container according to embodiment 15, wherein the container material comprises an organic polymer, preferably a polyethylene, a polypropylene, a cyclic olefin polymer or a cyclic olefin copolymer, or glass.

17. Container according to embodiment 15 or 16, wherein the container is a glass vial.

18. Container according to embodiment 15 or 16, wherein the container is a syringe, and wherein the syringe material preferably comprises a cyclic olefin copolymer.

19. Process for preparing an aqueous composition according to embodiments 1 to 12 comprising the steps of:

a. Dissolving a tonicity agent, preferably NaCl, in water,

b. Adding of HCl to adjust the pH to 1.6 to 2.0,

c. Adding and dissolving rocuronium,

d. Adding HCl to adjust the pH to 2.5 to 3.5,
and

e. Filling the composition into a container,

wherein sterility is brought about either by thermal sterilization or by aseptic filling.

20. The process according to embodiment 19, wherein the container is a glass vial and sterility is achieved by thermal sterilization.

21. The process according to embodiment 19, wherein the container is a glass vial and sterility is achieved by aseptic filling.

22. The process according to embodiment 19, wherein the container is a syringe, the syringe material preferably comprising a cyclic olefin copolymer, and wherein sterility is achieved by aseptic filling.

23. The process according to embodiment 20, wherein the thermal sterilization is performed at a temperature of 120° C. to 122° C. for 15 to 20 minutes.

24. The process according to embodiment 21 or 22, wherein aseptic filling comprises pre-sterilization of the composition and the syringe prior to filling the composition into the syringe.

25. The process according to embodiment 24, wherein the glass vial or the syringe is pre-sterilized via gamma irradiation or via treatment with ethylene oxide.

26. The process according to embodiment 24 or 25, wherein the composition is pre-sterilized through at least one, preferably through at least two filters with a pore size of 0.22 μm or less prior to filling the composition into the glass vial or the syringe.

27. The process according to any of embodiments 24 to 26, wherein filtering the composition and pre-sterilizing the glass vial or the syringe are performed in a sterile chamber.

### EXAMPLES

### Example 1a Preparation of the Composition According to the Invention

8.0 g sodium chloride were dissolved in 900 mL water for injection (WFI). 15.5 mL of 1M hydrochloric acid were added to adjust the pH to 1.8. Subsequently, 10 g of rocuronium bromide were added to the solution. The pH of the rocuronium-containing solution was adjusted to 3.0 by adding 1.6 mL of 1M hydrochloric acid. WFI was added to adjust the final volume to 1 L. The solution was filtered through a 0.2 μm filter. The filtrated solution was either thermally sterilized at 121° C. for 15 min in a vial or aseptically filled into a vial or into a syringe.

### Example 1b Alternative Preparation of the Composition According to the Invention

8.0 g sodium chloride were dissolved in 900 mL water for injection (WFI). 17.1 mL of 1M hydrochloric acid (15.5 mL+1.6 mL hydrochloric acid) were added directly to the initial solution of 8.0 g sodium chloride in 900 mL WFI. Subsequently, 10 g of rocuronium bromide were added to the solution. For final pH adjustment to 3.0, 1M hydrochloric acid or 1M sodium hydroxide were added. WFI was added to adjust the final volume to 1 L. The solution was filtered through a 0.2 μm filter. The filtrated solution was aseptically filled into a syringe.

The final composition comprises the following ingredients in a total volume of 1 L (Table 1):

TABLE 1
Final composition of the aqueous, room-temperature
stable composition of the present invention

| Ingredient: | 1 L formulation contains: | |
|---|---|---|
| Rocuronium Bromide | 10 | g |
| Sodium Chloride | 8.0 | g |
| HCl (1M) | 17.1 | mL |

| | |
|---|---|
| HCL (1M)/NaOH (1M) | q.s. pH = 3 |
| Water for injection | q.s. |

Several batches were prepared according to the above-described process. The final compositions had a titratable acidity of 8.5-10 mEq in glass vials and 19 mEq in syringes. The titratable acidity was measured at room temperature.

### Example 2 Stability of the Rocuronium Composition

a) pH Stability Upon Thermal Sterilization

The compositions prepared according to Example 1a were tested for pH-stability in glass vials upon thermal sterilization at 121° C. for 15 to 20 min. The pH before thermal sterilization was 3.0 and did not change upon thermal sterilization (Table 2). Further, no pH-changes were detected for compositions with initial pH of 2.8 and 3.2 (same preparation and composition as described in Example 1a except for the amount of HCl and NaOH to adjust the pH to 2.8 or 3.2 respectively). The pH of the aqueous compositions was measured at room temperature.

TABLE 2
pH of the compositions according to the present invention before
and after thermal sterilization at 121° C. for 15 to 20 min

| pH before thermal sterilization | pH after thermal sterilization |
|---|---|
| 2.8 | 2.8 |
| 3.0 | 3.0 |
| 3.2 | 3.2 |

b) Storage Stability

The compositions prepared according to Example 1 were filled into glass vials (10 mL (10R) glass vial with blowback, Schott AG, Example 1a) and in syringes (TopPac® Syringe 5 mL, Schott AG, Example 1b), respectively.

The filled vials and syringes were stored under different conditions for several months. The concentration of impurity C (the main degradation product/hydrolysis product of rocuronium bromide; des-17-acetyl rocuronium) was measured after 0 months, 1 month, 2 months, 3 months, 6 months, 9 months, and 12 months by HPLC:

b.1) Analytical Measurement of Impurity C

The analytical measurement of impurity C is based on the Pharm.Eu. monograph for Rocuronium Bromide (1764), Edition 10.3 and has been adapted to receive a maximum response of Rocuronium and the corresponding impurities:

Used Instruments:
Agilent 1290 UHPLC—DAD
Thermo Vanquish Horizon UHPLC— DAD

Used Column:
Nucleosil 100-5 OH (720143.46)

Used Chemicals:
Tetramethylammonium hydroxide; TMAH (p.a.)
Acetonitrile; ACN (HPLC grade)
Rocuronium Bromide (PharmEU or USP)
Rocuronium Bromide for peak identification (PharmEu)

TABLE 3
Instrument method description

| Column | Nucleosil 100-5 OH (720143.46) | | | | |
|---|---|---|---|---|---|
| Mobile Phase | 90% ACN with 10% TMAH Buffer at pH 7.4 | | | | |
| Autosampler Wash | 90% ACN | | | | |
| Elution condition | Isocratic | | | | |
| Flow | 2 | ml/min | | | |
| Column oven temp. | 25° | C. | | | |
| Autosampler temp. | 20° | C. | Preventing salt precipitation at | | |
| | | | lower temperatures due to the 95% | | |
| | | | ACN as sample solvent | | |
| Injection volume | 20 | μl | | | |
| Detection | 210 | nm | 1 nm Bandwidth | 5 Hz | Ref. wavelength |
| wavelength | | | | | “OFF” |

| | | |
|---|---|---|
| Slit | 8 | nm |
| Flow cell | 60 | mm |

Preparation of Mobile Phase:
A 0.025 M Tetramethylammonium hydroxide (TMAH) solution was prepared and adjusted to pH 7.4 with phosphoric acid.
9 volumes of acetonitrile were added to 1 volume of the 0.025 M TMAH solution, followed by stirring and sonicating. Subsequently, the solution was adjusted to room temperature.
Afterwards, the column was equilibrated for at least 20 minutes at a 2 ml/min flowrate.

Standard Preparation:
50 mg Rocuronium Bromide were dissolved in 10 ml of 90% acetonitrile (premixed by 9 volumes acetonitrile and 1 volume MilliQ water).
5 standards of about between 120% and 80% of API in final samples (approx. 4 mg/ml) and 9 standards between 3% and 0.01% of API were prepared.

Sample preparation:
A single replicate per sample was prepared as followed:
Sample container was mixed thoroughly and 400 μl were transferred into a 2 ml amber glass vial followed by evaporation under N₂ at about 1.5 bar (increased in 0.5 bar increments if necessary).
After evaporation, 50 μl MilliQ water were added and mixed, followed by addition of and mixing with 950 μl ACN. Precipitants were centrifuged at 3000 rpm at 4° C. for 2 min. The supernatant was transferred into a new 2 ml amber vial, screwed tightly and the vial was placed in the autosampler for analysis.

Analysis
The calibration standards were injected in ascending concentration in order to reduce possible carry over effects followed by the Rocuronium Bromide for peak identification solution. Afterwards a solvent blank and the samples were injected via single injection.

Data Processing
Rocuronium was determined by the received calibration curve of Rocuronium in the processed sample (approx. 4 mg/ml).
The Impurities were evaluated with the calibration curve established with the standards between 3%— 0.01% of Rocuronium in the processed sample.
The relative concentration was calculated by dividing the amount of each individual known Impurity by the amount of Rocuronium of the sample to receive the relative amount per Impurity in percent.

$$rel . {amount}_{Impurity} [ \% ] = \frac{A m o u n {t}_{Impurity} [ mg / ml ]}{A m o u n {t}_{R o c u r o n i u m} [ mg / ml ]} * 100$$

b.2) Long-Term Stability of Rocuronium Formulation

The different storage conditions for testing long-term stability were as follows:
a. Rocuronium formulation in glass vial; thermally sterilized; pH 3; storage at 25° C.
b. Rocuronium formulation in glass vial; thermally sterilized; pH 3; storage at 40° C.
c. Rocuronium formulation in glass vial; aseptically filled; pH 3; storage at 25° C.
d. Rocuronium formulation in glass vial; aseptically filled; pH 3; storage at 40° C.
e. Rocuronium formulation in glass vial; thermally sterilized; pH 3; storage at 30° C.
f. Rocuronium formulation in glass vial; aseptically filled; pH 3; storage at 30° C.
g. Rocuronium formulation in syringe; aseptically filled; pH 3; storage at 25° C.
h. Rocuronium formulation in syringe; aseptically filled; pH 3; storage at 40° C.
i. Rocuronium formulation in glass vial; aseptically filled; pH 2.8; storage at 25° C.
j. Rocuronium formulation in glass vial; thermally sterilized; pH 2.8; storage at 25° C.
k. Rocuronium formulation in glass vial; thermally sterilized; pH 2.8; storage at 30° C.
l. Rocuronium formulation in glass vial; aseptically filled; pH 2.8; storage at 40° C.
m. Rocuronium formulation in glass vial; thermally sterilized; pH 2.8; storage at 40° C.
n. Rocuronium formulation in glass vial; aseptically filled; pH 3.2; storage at 25° C.
o. Rocuronium formulation in glass vial; thermally sterilized; pH 3.2; storage at 25° C.
p. Rocuronium formulation in glass vial; thermally sterilized; pH 3.2; storage at 30° C.
q. Rocuronium formulation in glass vial; aseptically filled; pH 3.2; storage at 40° C.
r. Rocuronium formulation in glass vial; thermally sterilized; pH 3.2; storage at 40° C.

TABLE 4
Amount of rocuronium-related impurity C for rocuronium
formulations stored under different storage conditions

| | Impurity C | Impurity C | Impurity C | Impurity C | Impurity C | Impurity C |
|---|---|---|---|---|---|---|
| Storage | (%) after | (%) after | (%) after | (%) after | (%) after | (%) after |
| condition | 0 months | 1 month | 3 months | 6 months | 9 months | 12 months |
| a | 0.21 | 0.24 | 0.41 | 0.47 | 0.58 | 0.67 |
| b | 0.21 | 0.44 | 0.81 | 1.41 | | |
| c | 0.09 | 0.13 | 0.21 | 0.36 | | 0.53 |
| d | 0.09 | 0.30 | 0.70 | 1.29 | | |
| e | 0.17 | 0.25 | 0.47 | 0.65 | 0.83 | 1.02 |
| f | 0.09 | 0.17 | 0.31 | 0.56 | | 0.92 |
| g | 0.10 | 0.14 | 0.22 | 0.33 | | 0.52 |
| h | 0.10 | 0.26 | 0.67 | 1.31 | | |
| i | * | 0.16 | 0.19 | 0.30 | | 0.54 |
| j | * | 0.23 | 0.26 | 0.36 | 0.55 | 0.57 |
| k | * | 0.24 | 0.35 | 0.55 | 0.82 | 1.02 |
| l | * | 0.28 | 0.64 | 1.20 | | |
| m | * | 0.38 | 0.69 | 1.29 | | |
| n | 0.08 | 0.18 | 0.21 | 0.35 | | 0.62 |
| o | 0.19 | 0.29 | 0.31 | 0.50 | 0.63 | 0.76 |
| p | 0.19 | 0.33 | 0.45 | 0.75 | 1.05 | 1.28 |
| q | 0.08 | 0.37 | 0.82 | 1.56 | | |
| r | 0.19 | 0.48 | 0.95 | 1.74 | | |

* (means below the LOQ—limit of quantitation)

The impurity concentrations for samples with initial pH 3.0 stored at 25° C. (samples a, c and g) were measured for 12 months (Table 4). These impurity concentrations were used to extrapolate the time until impurity C concentration would reach a threshold value of 2.5% (estimated shelf-life of the composition according to the present invention).

Accordingly, the composition according to the invention has an estimated shelf-life of up to 59.5 months, i.e. approximately 5 years, when stored at 25° C. after thermal sterilization (storage condition a.). Even when stored at an elevated temperature of 30° C., the composition according to the present invention has an expected shelf-life of up to 32.5 months after thermal sterilization (storage condition e). Thus, the aqueous composition of the invention has a shelf-life of at least 2 years, for example at least 2.5 years, at least 3 years, or at least 4 years, when stored at room temperature.

Storage stability tests have also been performed for compositions according to the present invention with a pH of 2.8 and 3.2 (storage conditions i to r). Storage stability and extrapolated shelf-life of these compositions were comparable to those with a pH of 3.0.

b.3) pH Stability of Rocuronium Formulation Upon Storage

The pH of the Rocuronium formulations under different storage conditions a. to r. as described in Example b.2) were measured for 12 months (Table 5).

TABLE 5
pH values for rocuronium formulations stored under different storage conditions

| Storage | pH after 0 | pH after 1 | pH after 3 | pH after 6 | pH after 9 | pH after 12 |
|---|---|---|---|---|---|---|
| condition | months | month | months | months | months | months |
| a | 3.0 | 3.0 | 3.0 | 3.0 | 3.1 | 3.1 |
| b | 3.0 | 3.0 | 3.0 | 3.0 | | |
| c | 3.0 | 3.0 | 3.0 | 3.0 | | 3.0 |
| d | 3.0 | 3.0 | 3.0 | 3.0 | | |
| e | 3.0 | 3.1 | 3.1 | 3.0 | | 3.0 |
| f | 3.0 | 3.1 | 3.1 | 3.0 | | 3.0 |
| g | 3.0 | 3.1 | 3.0 | 3.0 | | 3.0 |
| h | 3.0 | 3.1 | 3.0 | 3.0 | | |
| i | 2.8 | 2.8 | 2.8 | 2.8 | | 2.8 |
| j | 2.8 | 2.8 | 2.8 | 2.8 | 2.9 | 2.8 |
| k | 2.8 | 2.8 | 2.8 | 2.8 | 2.9 | 2.8 |
| l | 2.8 | 2.8 | 2.8 | 2.8 | | |
| m | 2.8 | 2.8 | 2.8 | 2.8 | | |
| n | 3.2 | 3.2 | 3.2 | 3.2 | | 3.2 |
| o | 3.2 | 3.2 | 3.2 | 3.2 | 3.3 | 3.2 |
| p | 3.2 | 3.2 | 3.2 | 3.3 | 3.3 | 3.3 |
| q | 3.2 | 3.2 | 3.3 | 3.3 | | |
| r | 3.2 | 3.2 | 3.3 | 3.3 | | |

The pH of the aqueous, room-temperature stable composition of the present invention did not drift upon storage by more than 0.1 pH units.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An aqueous, room-temperature stable composition comprising rocuronium, wherein the composition has a pH of 2.5 to 3.5, and the composition has a titratable acidity of not more than 35 mEq.

2. The composition according to claim 1, wherein the drift of the pH following sterilization, or following storage of the composition at room temperature, is not more than 0.5 pH units.

3. The composition according to claim 1, wherein the drift of the pH following sterilization, or following storage of the composition at room temperature, is not more than 0.2 pH units.

4. The composition according to claim 1, wherein the drift of the pH following sterilization, or following storage of the composition at room temperature, is not more than 0.1 pH units.

5. The composition according to claim 1, wherein the composition does not exhibit more than 0.7% rocuronium-related impurity C after storage for 6 months at room temperature.

6. The composition according to claim 1, wherein the composition does not exhibit more than 1.1% rocuronium-related impurity C after storage for 12 months at room temperature.

7. The composition according to claim 1, wherein the composition does not exhibit more than 1.0% rocuronium-related impurity C after storage for 6 months at 30° C.

8. The composition according to claim 1, wherein the composition does not comprise a buffer.

9. The composition according to claim 1, wherein the composition has a titratable acidity of not more than 30 mEq.

10. The composition according to claim 1, wherein the composition has a titratable acidity of not more than 20 mEq.

11. The composition according to claim 1, wherein the rocuronium comprises rocuronium bromide.

12. A method for facilitating tracheal intubation in a patient, the method comprising administering to the patient the composition according to claim 1.

13. The method of claim 12, wherein the method comprises providing general anesthesia to facilitate tracheal intubation during routine sequence induction or to provide skeletal muscle relaxation during surgery or to facilitate tracheal intubation during rapid sequence induction or for short term use in the intensive care unit.

14. A container comprising the composition according to claim 1.

15. The container according to claim 14, wherein the container material comprises an organic polymer or glass.

16. The container according to claim 15, wherein the container is a glass vial.

17. The container according to claim 15, wherein the container is a syringe.

18. A process for preparing an aqueous composition according to claim 1, the method comprising:
a. Dissolving a tonicity agent in water,
b. Adding of HCl to adjust the pH to 1.6 to 2.0,
c. Adding and dissolving rocuronium,
d. Adding HCl to adjust the pH to 2.5 to 3.5, and
e. Filling the composition into a container, wherein sterility is brought about either by thermal sterilization or by aseptic filling.

19. The process according to claim 18, wherein the drift of the pH following sterilization, or following storage of the composition at room temperature, is not more than 0.5 pH units.

20. The process according to claim 18, wherein the drift of the pH following sterilization, or following storage of the composition at room temperature, is not more than 0.2 pH units.
